(19) United States
(12) Patent Application Publication
(10) Pub. No.: US 2004/0109876 A1
(43) Pub. Date: Jun. 10, 2004

(54) Vaccine composition, HIV-infection suppression factor and method for the vaccination against HIV

(75) Inventors: Naoki Yamamoto, Oota-ku (JP); Yuetsu Tanaka, Naha-shi (JP); Masataka Nakamura, Suginami-ku (JP); Kunitaka Hirose, Nerima-ku (JP)

Correspondence Address: BOZICEVIC, FIELD & FRANCIS LLP, 200 MIDDLEFIELD RD, SUITE 200, MENLO PARK, CA, 94025, US

(73) Assignee: Kureha Chemical Industry Co., Ltd.

(21) Appl. No.: 10/634,743

(22) Filed: Aug. 4, 2003

Related U.S. Application Data
(60) Provisional application No. 60/429,213, filed on Nov. 25, 2002.

(51) Int. Cl. A61K 39/21

(52) U.S. Cl. 424/208.1

(57) ABSTRACT

A possibility of a dendritic cell (DC)-based vaccination against HIV-1 infection in humans was explored in SCID mice reconstituted with human peripheral blood mononuclear cells (PBMC). HIV-1-negative normal human PBMC were transplanted into the spleens of SCID mice (hu-PBL-SCID-spl) together with autologous mature DC pulsed with either inactivated HIV-1 (R5 or X4 strain) or ovalbumin (OVA), followed by a booster injection with autologous DC pulsed with respective antigens after 5 days. Five days later, these mice were challenged ip. with R5 HIV-1_{JR-CSF}. Analysis of infection on seven days post infection showed that the DC-HIV-1-immunized hu-PBL-SCID-spl mice, irrespective of immunized HIV-1 strains, were protected against the HIV-1 infection. In contrast, none of the DC-OVA-immunized mice were protected. Sera from the DC-HIV-1-, but not DC-OVA-, immunized mice interfered with in vitro infection of activated PBMC and macrophages with R5, but not X4, HIV-1. Upon restimulation with HIV-1 in vitro, the human CD4+ T cells derived from the DC-HIV-1-immunized mice produced similar R5 HIV-1 suppression factor. Neutralizing antibodies against human RANTES, MIP-1-alpha, MIP-1-beta, IFN-alpha, IFN-beta, IFN-gamma, IL-4, IL-10, IL-13, IL-16, MCP-1, MCP-3, TNF-alpha or TNF-beta did not reverse the HIV-1 suppressive activity. These results show that inactivated HIV-1-pulsed autologous DC can stimulate human CD4+ T cells living in the hu-PBL-SCID-spl mice to produce unknown soluble factor(s) protective against R5 HIV-1 infection.

### FIELD OF THE INVENTION

This application claims the benefit of U.S. Provisional Application No. 60/429,213 filed Nov. 25, 2002. The present invention relates to a vaccine composition, a HIV-infection suppression factor and a method for the vaccination against HIV. More specifically, the vaccine composition and the method of the vaccination relate to a dendritic cell (DC) based vaccination using DC pulsed with inactivated human immunodeficiency virus (HIV).

### BACKGROUND OF THE INVENTION

Mice with severe combined immunodeficiency (SCID), when adoptively transferred with human peripheral blood mononuclear cells (PBMC), develop a surrogate human immune system. These mice, termed hu-PBL-SCID mice, have served as a valuable model for the study of human immunodeficiency virus type-1 (HIV-1) pathogenesis (1, 2). It has been shown that the human T cells transplanted into a SCID mouse are activated (3) and proliferate in response to mouse antigens presented by antigen presenting cells (APC) of mouse origin (4). Trials have been made to induce human immune responses in the hu-PBL-SCID mice (5-8). There are two major limitations to developing strong human immune responses in the hu-PBL-SCID mice. The first may be due to the lack of appropriate human APC, including DC, while the second is the lack of a suitable microenvironment, such as normal lymphoid organs (4). Both may facilitate primary interaction between T cells and APC. To overcome the lack of APC, Delhem et al. (9) have used autologous skin containing tissue DC as APC sources and have succeeded in induction of primary MHC-restricted human T cell responses against HIV-1 envelope in the hu-PBL-SCID mice. Furthermore, Santini et al. (10) have recently reported that HIV-1-pulsed, monocyte-derived human mature DC can stimulate primary human anti-HIV-1 antibody production in the SCID mouse system.

Because the hu-PBL-SCID mice are permissive for macrophage-tropic R5 HIV-1 (11), this animal model should provide us with valuable information in evaluating HIV-1 vaccine candidates. Despite success in the induction of such human T and B cell immune responses against HIV-1, it has not been possible to induce a protective immunity against HIV-1 in this animal model. In the present study, we found that transfer of PBMC, together with inactivated HIV-1-pulsed DC, directly into the mouse spleen elicited a protective immune factor against R5 HIV-1 infection. The factor was mainly produced by human CD4+ T cells in response to HIV-1, and is unrelated to known R5 HIV-1 suppressive cytokines. The present results implicate a new concept on a DC-based HIV-1 vaccination in humans.

### SUMMARY OF THE INVENTION

The present invention provides a vaccine composition comprising autologous mature DC pulsed with inactivated HIV.

The present invention also provides an HIV-infection suppression factor which is produced by human CD4+ pulsed with inactivated HIV, has a molecule weight of more than 100 kDa, is not absorbed to heparin-Sepharose columns, and is inactivated by heating at 56 degree Celsius for 30 min.

Further, the present invention provides a method for the vaccination against HIV comprising: collecting PBMC from a subject to be vaccinated, culturing the PBMC in medium containing GM-CSF and human IL-4 to prepare autologous mature DC, pulsing the DC with inactivated HIV, and administering the DC to the subject.

### BRIEF DESCRIPTION OF THE DRAWINGS

FIGS. 1A, 1B and 1C. are the graphs showing induction of antigen-specific human immune responses in the hu-PBL-SCID-spl mice by immunization with antigen-pulsed DC. (FIG. 1A) PBMC (3×10⁶ cells) only (non-immune), PBMC and OVA 100 μg (OVA-immune), or PBMC and DC (5×10⁵ cells) pulsed with OVA 100 μg (DC-OVA immune) were engrafted into the spleens of SCID mice. Five days later, mice were received a booster injection with OVA or DC-OVA. Then 5 days later, serum samples were collected, and human anti-OVA antibodies were measured by ELISA. *p<0.05

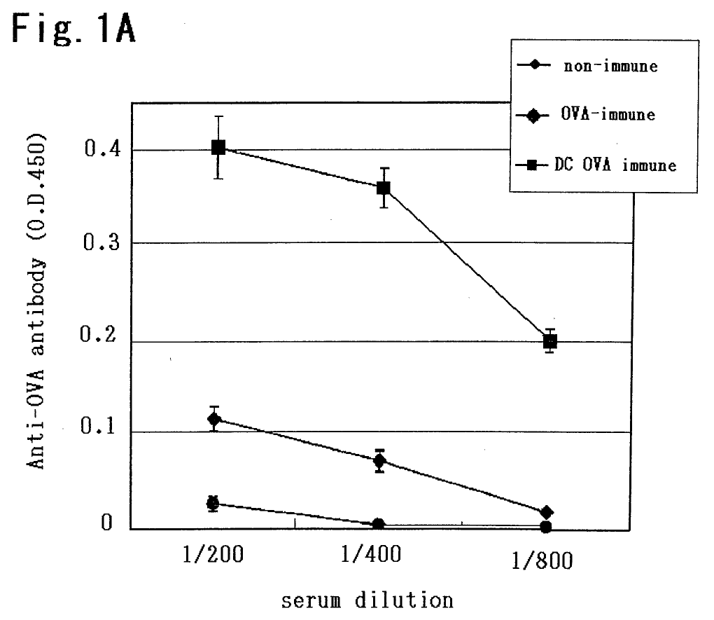

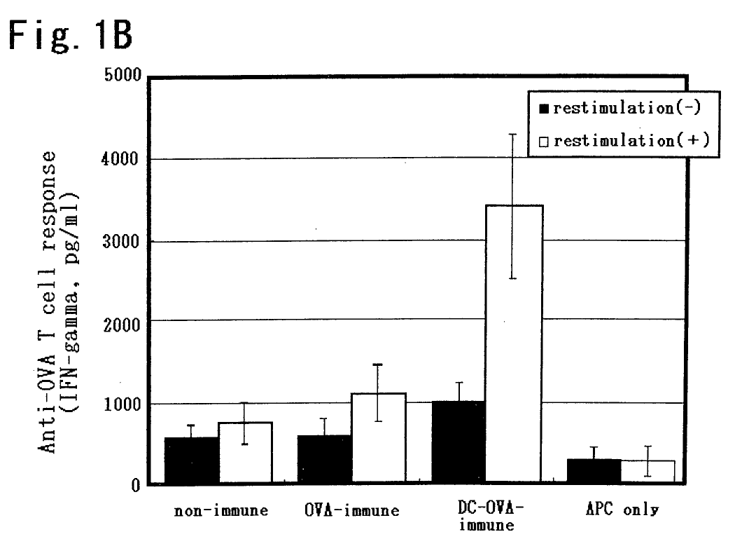

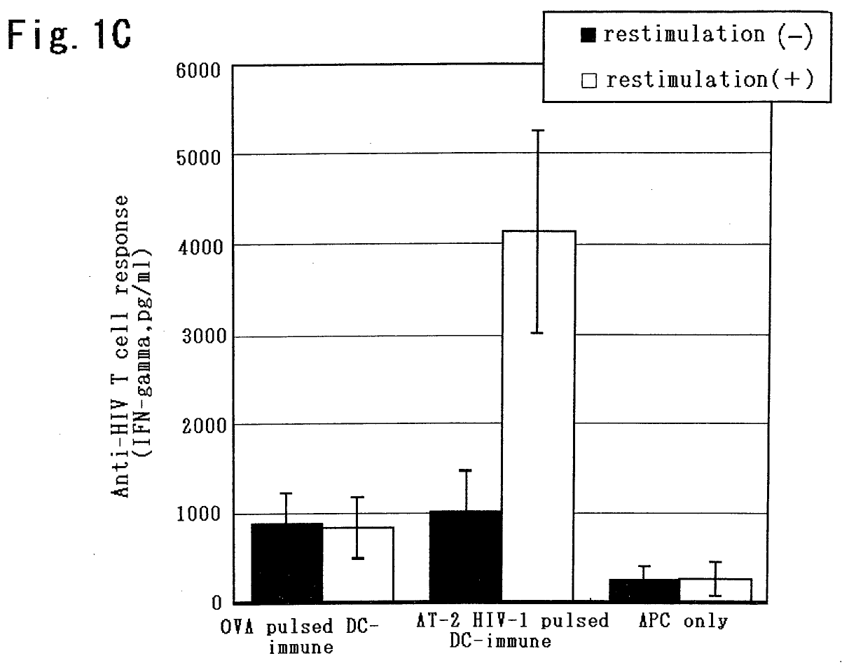

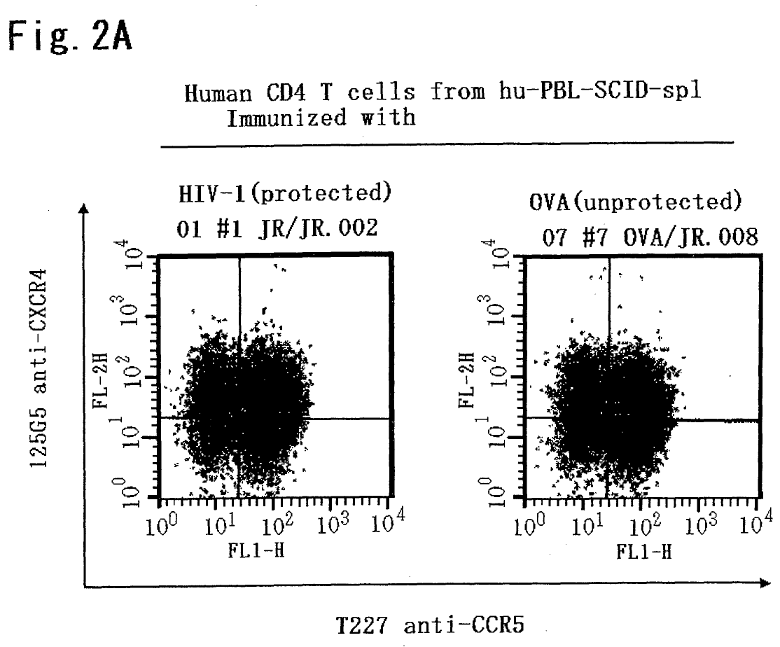

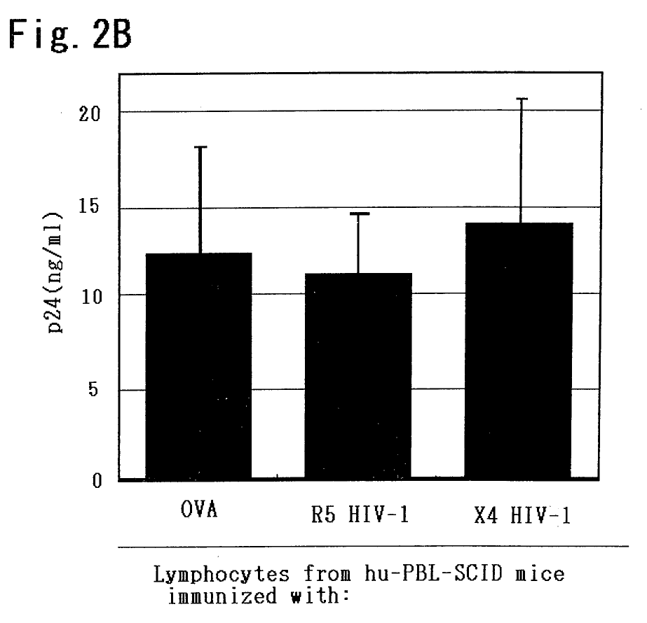

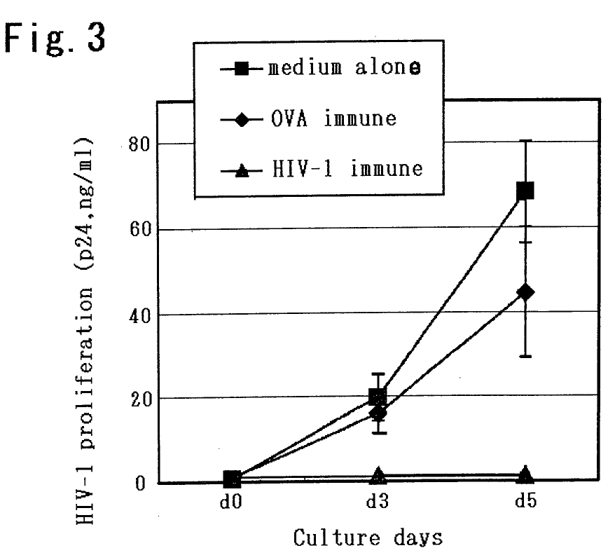

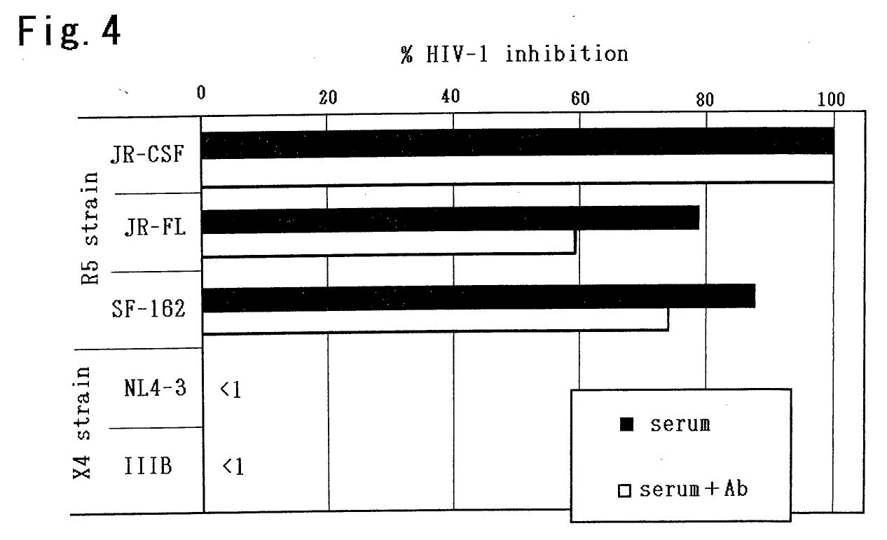

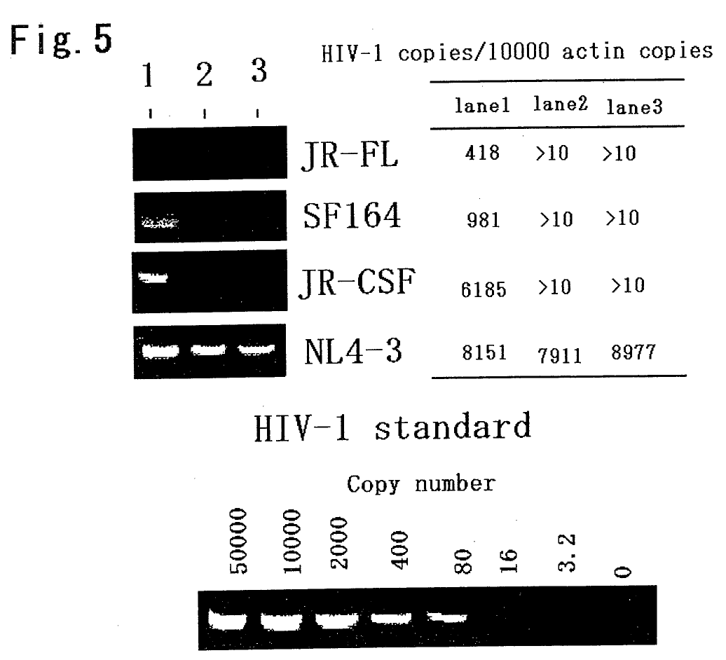

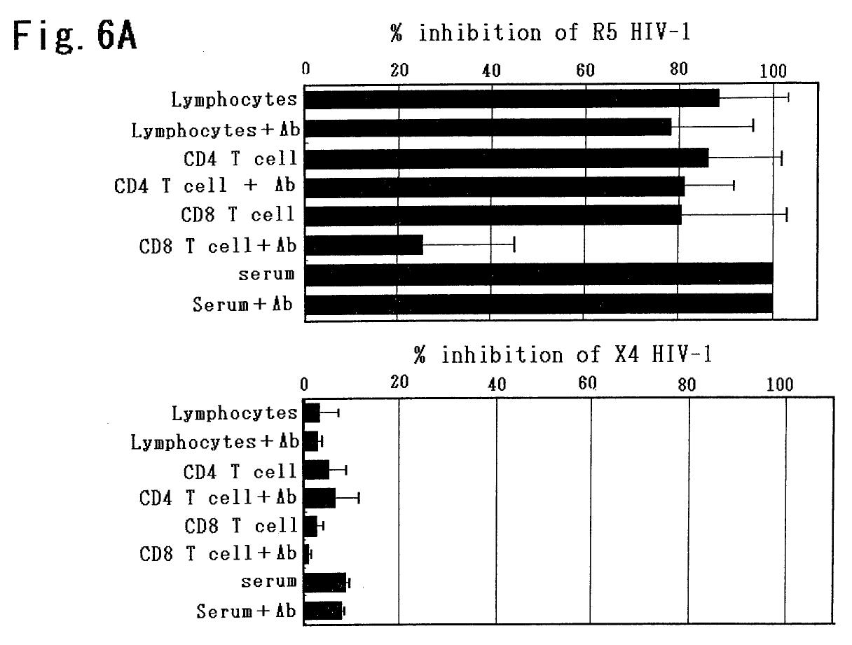

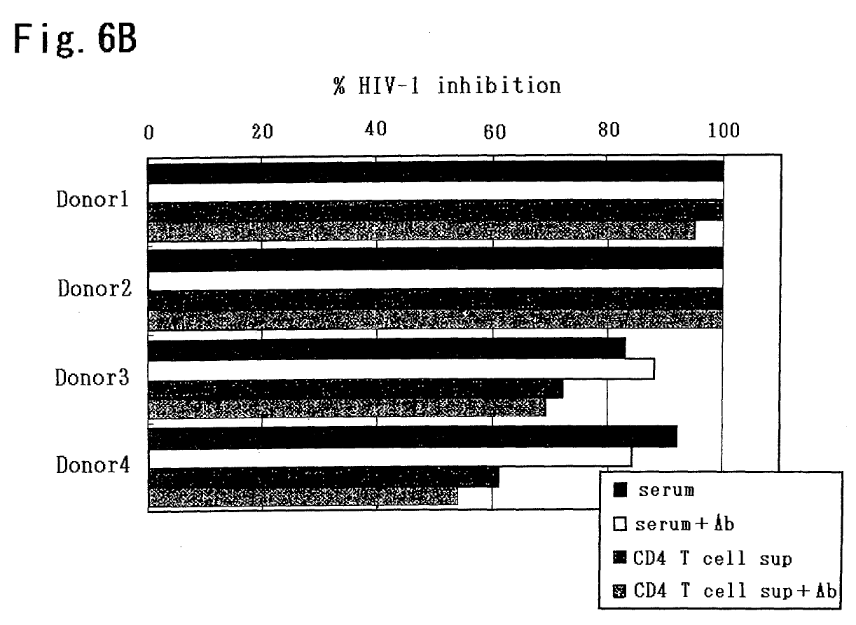

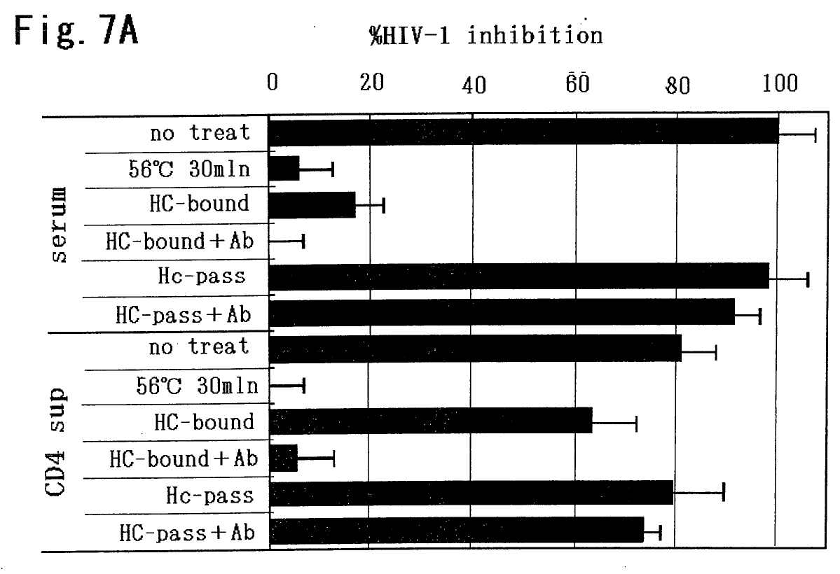

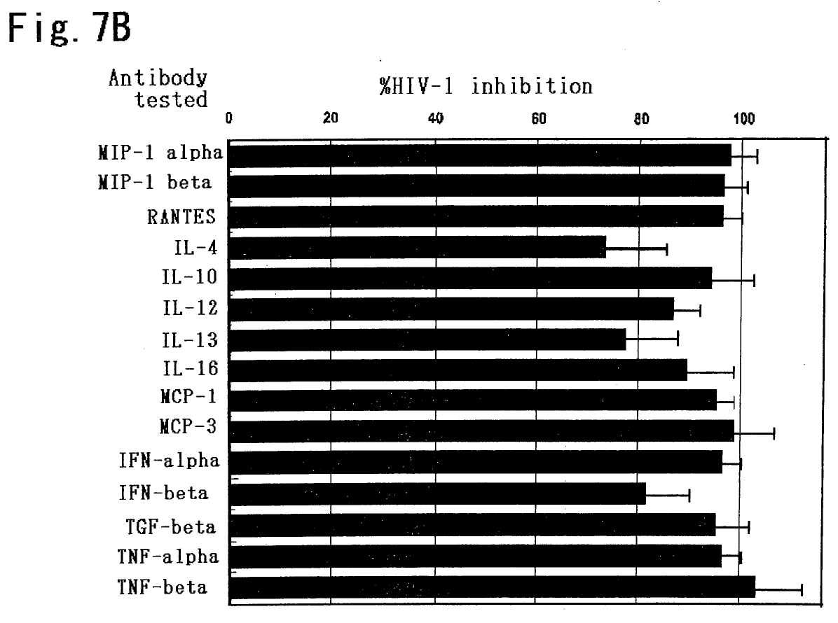

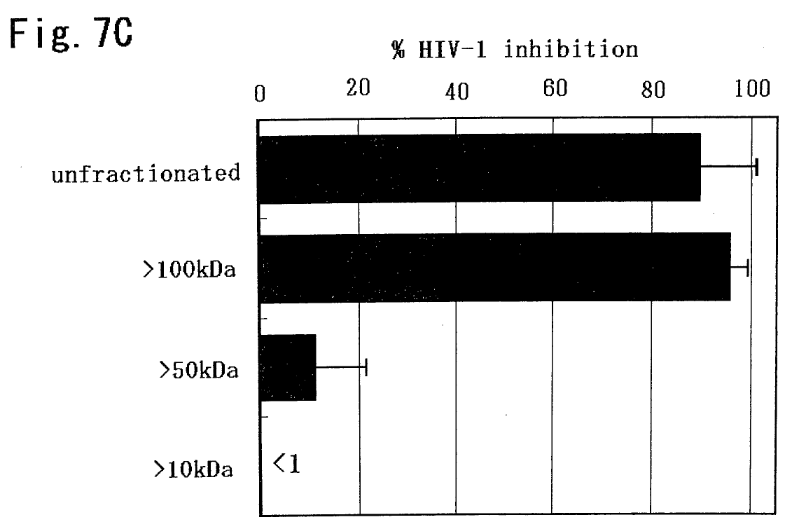

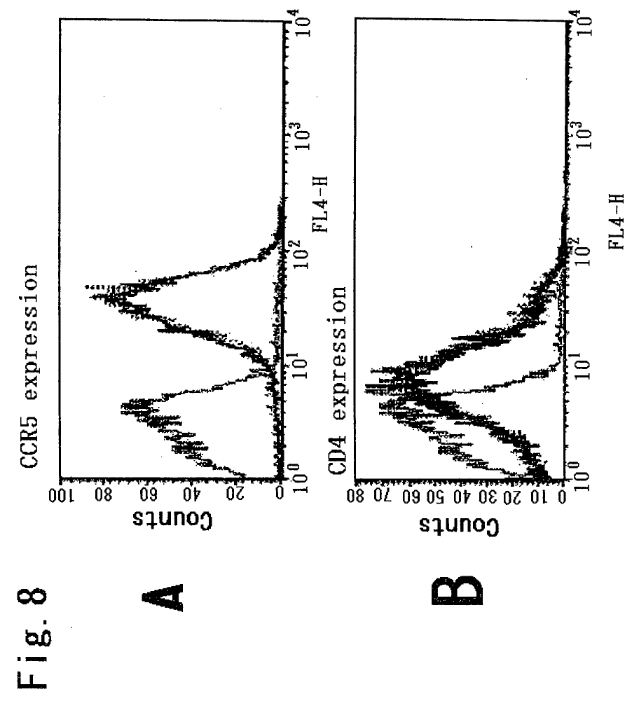

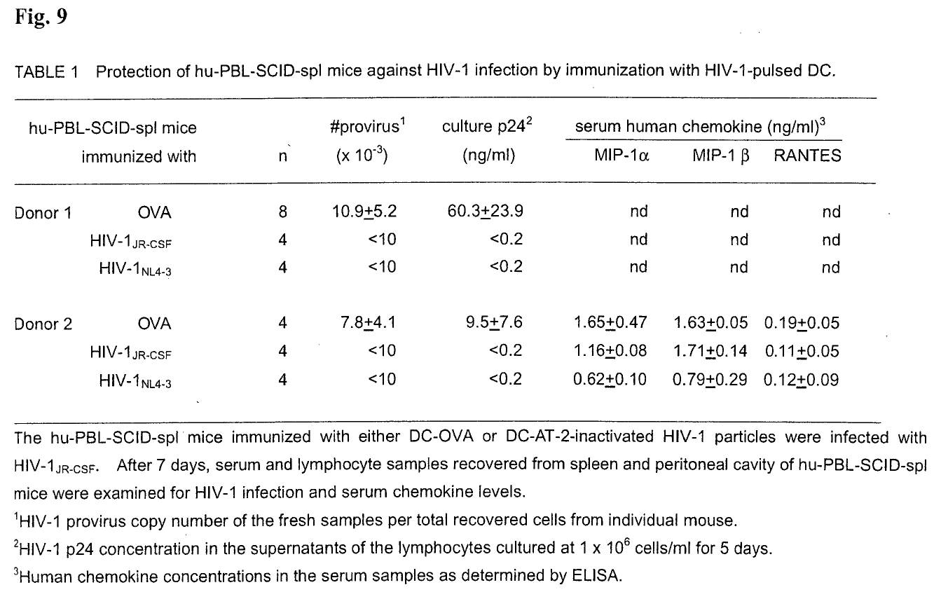

(FIG. 1B) Lymphocytes (2×10⁶ cells) recovered from the hu-PBL-SCID-spl mice were cultured with 2×10⁵ autologous APC (adherent cells of PBMC) in the presence (restimulation (+)) or absence (restimulation (−)) of 1 μg/ml OVA at 37 ° C. for 2 days in 1 ml 20 U/ml human IL-2-containing RPMI medium. APC only, without lymphocytes from the mice. *p<0.05.

(FIG. 1C) Lymphocytes (2×10⁶ cells) recovered from the hu-PBL-SCID-spl mice immunized with either DC-OVA or DC-AT-2 inactivated HIV-1_{JR-CSF} were restimulated as (B) in the presence of OVA or AT-2 inactivated HIV-1 (40 ng p24), respectively, for 2 days. Concentrations of human IFN-gamma in the culture supernatants were determined by ELISA. All results are expressed as mean ± SD from six independent experiments. *p<0.05.

FIG. 2A illustrates human CD4+ T cells from HIV-1-protected hu-PBL-SCID-spl mice express CCR5 and are permissive for R5 HIV-1 infection in vitro.

(FIG. 2A) Lymphocytes recovered from the hu-PBL-SCID-spl mice immunized with DC-OVA, or DC-HIV-1_{JR-CSF}, were stained with anti-CD4, anti-CCR5 and anti-CXCR4. The expression profiles of CCR5 and CXCR4 on the CD4+ T cells are shown. Representative data of four independent experiments.

FIG. 2B is the graph showing the number of lymphocytes from hu-PBL-SCID mice immunized with OVA, R5 HIV-1 or X4 HIV-1.

(FIG. 2B) Lymphocytes recovered from the hu-PBL-SCID-spl mice immunized with DC-OVA, DC-HIV-1_{JR-CSF}, or DC-HIV-1_{NL}4-3 were washed, and infected with 500 TCID₅₀ HIV-1_{JR-CSF} at 37° C. for 4 h. After washing, the cells were cultured for 5 days in 20 U/ml IL-2-containing RPMI medium. HIV-1 p24 produced in culture supernatants were measured by ELISA. Representative data of three independent experiments.

FIG. 3 is the graph showing inhibition of R5 HIV-1 infection by the HIV-1 immune serum. Activated PBMC (5×10⁵ cells/well) were incubated in medium or final 20% serum samples obtained from either DC-OVA immune or DC-HIV-1_{JR-CSF} immune hu-PBL-SCID-spl mice at 37° C. for 1 h. Without washing, the PBMC were infected with 500TCID₅₀ HIV-1 _{JR-CSF} at 37° C. for 4 h. After washing, cells were incubated in IL-2-containing medium for indicated periods. Proliferation of HIV-1 was monitored by quantitating HIV-1 p24 in the culture supernatants. Representative data of three independent experiments.

FIG. 4 is the graph showing inhibition of R5, but not X4, HIV-1 by the HIV-1 immune serum. Activated PBMC were treated with 10% pooled serum samples obtained from DC-HIV-1_{JR-CSF} immune hu-PBL-SCDI-spl mice at 37° C. for 1 h. Then the PBMC were infected with 500 TCID₅₀ HIV-1 (the R5 stains JR-CSF, JR-FL and SF162, and the X4 strains NL4-3 and IIIB) at 37° C. for 4 h. After washing, cells were incubated in IL-2-containing medium for 5 days, and HIV-1 p24 in the culture supernatants was determined. The HIV-1 suppressive activities of the serum preincubated with a mixture of anti-beta chemokine antibodies (+Ab) were also shown. Percent inhibition was calculated against the medium controls: JR-CSF, 18.7 ng/ml; JR-FL, 7.6 ng/ml; SF162, 6.6 ng/ml; NL4-3, 18.3 ng/ml; and IIIB, 10.2 ng/ml. Results are expressed as the mean ±SD from six independent experiments.

FIG. 5 is the graph showing blocking of R5 HIV-1 infection in macrophage. Cultured were preincubated in medium (lane 1), the pooled serum samples from DC-HIV-1_{JR-CSF} immune SCID mice in the absence (lane 2) or presence (lane 3) of a mixture of the anti-β chemokines (Ab), and then infected with R5 HIV-1 strains (JR-FL, SF-164 and JR-CSF) or X4 HIV-1_{NL}4-3. After washing, the cells were cultured for 2 days and cellular DNA were proved for the copy number of HIV-1 provirus and actin. The estimated HIV-1 copy numbers per 10,000 actin were shown in the right. Representative data of three independent experiments.

FIG. 6A is the graph showing % inhibition of R5 HIV-1 and X4 HIV-1 in each cell population producing the suppression factor.

(FIG. 6A) Lymphocytes from DC-HIV-1_{JR-CSF} immune hu-PBL-SCID-spl mice were positively separated into human CD4+ T and CD8+ T cell subpopulation by a magnet beads method. Non-separated (lymphocytes), CD4+ T and CD8+ T cells (2×10⁶ cells) were cultured with autologous APC (2×10⁵ cells) in the presence of AT-2 inactivated HIV-1 (40 ng p24) for 2 days in 1 ml IL-2-containing medium. Activated PBMC were pretreated with these culture supernatants (final 50% concentration) in the absence or presence (+Ab) of a mixture of anti-beta chemokine neutralizing antibodies, and then infected with either 500 TCID₅₀ HIV-1_{JR-CSF} or HIV-1N_{L}4-3. After washing, PBMC were cultured for 5 days, and HIV-1 p24 produced in the culture supernatants was measured. DC-HIV-1 immune serum (20%) was used as a positive control. The percent inhibition was calculated against the medium controls, 19.4 ng/ml for JR-CSF, and 25.9 ng/ml for NL4-3.

FIG. 6B is the graph showing % HIV-1 inhibition in each donor. (B) Sera (20%) and culture supernatants (50%) of restimulated CD4+ T cells from the DC-HIV-1_{JR-CSF}-immune hu-PBL-SCID-spl mice reconstituted with PBMC and DC from four different donors were examined for the suppressive activity against HIV-1_{JR-CSF} infection of PBMC. Percent HIV-1 inhibition was calculated against the medium control, p24 value of 23.4 ng/ml.

FIG. 7A is the graph showing partial characterization of the HIV-1 suppression factor.

(FIG. 7A) The immune serum and the in vitro restimulated culture supernatants from DC-HIV-1_{JR-CSF} immune hu-PBL-SCID-spl mice were heated, or separated into heparin-binding and -unbinding fractions by passing the heparin-Sepharose column. The heparin-bound fraction was eluted in 2 M NaCl buffer. PBMC were pretreated with these samples (at final 20% serum and 50% culture supernatants) in the absence or presence (+Ab) of the mixture of anti-beta chemokine antibodies, infected with 500 TCID₅₀ HIV-1_{JR-CSF}, and then cultured for 5 days. The percent inhibition was calculated against the medium control, p24 value of 22.3 ng/ml.

FIG. 7B is the graph showing % HIV inhibition in antibody test of the HIV-1 suppression factor.

(FIG. 7B) The pooled sera (10%) from the DC-HIV-1_{JR-CS}F-immune hu-PBL-SCID-spl mice were preincubated with each anti-human cytokine antibody at 10 μg/ml, and then were examined for the suppression activity against HIV-1_{JR-CSF} infection of PBMC.

FIG. 7C is the graph showing % HIV inhibition of the HIV-1 suppression factor in each molecular weight fraction.

(FIG. 7C) The pooled sera from the DC-HIV-1_{JR-CSF}-immune hu-PBL-SCID-spl mice were passed through the heparin Sepharose column and then serially filtrated through the Centricon with 100, 50 and 10 kDa cut-off filters. The fractions were examined for the suppressive activity against HIV-1_{JR-CSF} infection of PBMC at 10% concentration. Representative data of four independent experiments.

FIG. 8 is the graph showing no effect of the suppression factor on CCR5 and CD4 expression on macrophages. Macrophages cultured for 5 days in M-CSF were treated with either the serum (10%) or the in vitro restimulated CD4+ T cell culture supernatants (50%) originated from the DC-HIV-1_{JR-CSF} immune hu-PBL-SCID mice at 37° C. for 1 h. Then cells were FcR-blocked and stained with anti-CCR5 (Panel A) and anti-CD4 (Panel B). Representative data of four independent experiments.

FIG. 9 is Table 1 showing levels of beta-chemokines in sera from the DC-OVA-immunized mice.

### DETAILED DESCRIPTION OF THE PREFERRED EMBODIMENTS

(Materials and Methods)

Mice

SCID mice used were C.B-17-scid mice were purchased from Crea Japan (Kanagawa, Japan). NOD/Shi-scid, cg^{-/-} (12) and BALB/c-rag2^{-/-} gc^{-/-} mice (13) were also used in the study. The mice were kept in the SPF and P3 animal facilities at the Laboratory Animal Center, University of the Ryukyus. The committee on animal research of University of the Ryukyus approved protocols for the care and use of hu-PBL-SCID mice. Natural killer cells in the C.B-17-scid mice were depleted by i.p. injection of 1 mg/animal rat anti-mouse IL-2R-beta (TM-beta-1) (14).

Reagents

RPMI 1640 medium (SIGMA, St. Louis, Mo.) supplemented with 5% fetal calf serum, 100 U/ml of penicillin and 100 μg/ml of streptomycin (hereinafter called RPMI medium), Iscove's modified Dulbecco's medium (Lifetechnology, NY) supplemented with 10% fetal calf serum with the antibiotics (hereinafter called Iscove's medium) were used. Neutralizing monoclonal antibodies (mAb), anti-human MIP-1α, human MIP-1β, human IL-4, human IL-10, human IL-12, human IL-13, human IL-16, human MCP-1 and human MCP-3, were purchased from R&D systems (Rockville, Md.). Goat anti-human IFN-alpha and IFN-beta were purchased from PEPRO TECH EC LTD (London, UK). Human IL-4 and GM-CSF were generated in COS cell cultures transfected with expression plasmid DNA, pCMhIL4 and pCMhGM (RIKEN Gene Bank, Ibaraki, Japan), respectively, by the FuGENE6 method (Roche Diagnosis Corporation, Indianapolis, Ind.). Concentrations of human IL-4 and GM-CSF were determined using commercial ELISA kits (BioSouce, Camarillo, Calif.). Human recombinant IL-2 and M-CSF were purchased from Shionogi (Osaka, Japan) and PEPRO TEC, respectively.

Virus

HIV-1_{JR-CSF} and HIV-1_{JR-FL} (15) and HIV-1 _{NL}4-3 (16) were produced in 293T cell by transfection with HIV-1 infectious plasmid DNA by a calcium phosphate method (17). HIV-1_{SF}162 (18) was produced in phytohemagglutinin (PHA)-stimulated PBMC. HIV-1_{IIIB} was harvested from Molt-4/IIIB cells. The 50% tissue culture infectious dose (TCID₅₀) was determined by an endpoint infectious assay using PHA-activated PBMC. As immunogen, HIV-1 stocks were prepared in autologous PBMC cultures activated with anti-CD3 mAb. These HIV-1 preparations were inactivated with aldorichol-2 (AT-2), as described Rossio et al. (19). AT-2 was removed by ultrafiltration in phosphate-buffered saline (PBS) using 100-kDa cut-off centrifugal filtration device (Centriprep 100; Amicon, Beverly, Mass.) for 3 times.

Genera tion of Monocyte-Derived DC

Fresh PBMC at 5×10⁶ cells/ml in RPMI medium were plated on 12-well plates (1 ml/well), which had been coated with autologous plasma for 30 min at 37° C., and cultured at 37° C. for 1 h. After gentle washing with serum-free RPMI-1640 medium, the adherent cells were cultured in Iscove's medium (2 ml/well) containing human GM-CSF (500 ng/ml) and IL-4 (200 ng/ml) for 5 days. Immature DC cultures were depleted of contaminating lymphocytes using the monocyte negative isolation kit (Dynal, Oslo, Norway), and were further cultured in human IFN-58 (1,000 U/ml; Toray, Tokyo, Japan) for 1 day to obtain mature DC, after the method originally described by Santini et al. (10).

Transplantation, Immunization and Infection

Mature DC (5×10⁵ cells) were pulsed with AT-2 inactivated HIV-1 (40 ng p24) or 100 μg of OVA, for 2 h at 37° C. in 100 μl RPMI medium. These DC were mixed with autologous fresh PBMC (3×10⁶ cells) in a final volume of 100 μl, and then were transferred into the spleen of SCID mice. Five days later, the same number of DC pulsed with antigen were inoculated into the spleen or peritoneal cavity. Five days later, some mice were sacrificed and blood were collected by cardiocentesis and human lymphocytes were recovered from peritoneal lavage and the spleen. The other mice were challenged i.p. with 1000 TCID₅₀HIV-1_{JR-CSF} (100 μl/animal). After 7 days, serum and human lymphocytes from peritoneal lavage and spleen were collected. The peritoneal lavage fluids, sera and lymphocyte culture supernatants were examined for HIV-1 p24 by an ELISA kit (Zepto Metrix, Buffalo, N.Y.). Fresh lymphocytes were examined for proviral DNA by a quantitative PCR (20). Target cells used for in vitro infection assays were normal PBMC activated with magnetic beads conjugated with anti-CD3 and anti-CD28 mAb (Dynal, Oslo, Norway) at cell to bead ratio of 1:1 in 20 U/ml human IL-2-containing RPMI medium for 3 days. Some experiments utilized cultured macrophages derived from normal human PBMC, which were prepared by culturing adherent PBMC in 20 ng/ml human M-CSF for 5-7 days. These activated PBMC or cultured macrophages (5×10⁵ cells) were preincubated in 50 μl medium, diluted serum and culture supernatant samples at 37° C. for 1 h in 96 well U-bottom microtiter plates (BD Pharmingen, San Diego, Calif.). Then 50 μl HIV-1 stock containing 500 TCID₅₀ HIV-1 was added to each well. After incubation at 37° C. for 3 h, the cells were washed three times and cultured in 20 U/ml IL-2-containing RPMI medium for 3-5 days. HIV-1 proliferation was monitored by quantitating HIV-1 p24 produced in the culture supernatants. In order to determine the inhibitory effects of defined anti-human cytokine antibodies on the present HIV-1 suppression factor, the serum and the restimulated culture supernatants were preincubated with 10 μg/ml of cytokine neutralizing antibodies on ice for 30 min, and then applied to infection assays.

In Vitro Re-Stimulation

For the measurement of antigen specific human cellular immune responses, lymphocytes (2×10⁶ cells) collected from spleen and peritoneal lavage of the immunized mice were cultured with 2×10⁵ autologous APC (adherent cells of PBMC) in the presence or absence of either 1 μg OVA or AT-2 inactivated HIV-1 containing 40 ng p24 in the 24 well plates (BD Pharmingen, San Diego, Calif.) in 1 ml 20 U/ml human IL-2-containing RPMI medium at 37° C. for 2 days. The concentration of human IFN-gamma produced in the culture supernatants was determined by commercial ELISA kits (R&D Systems, Rockville, Md.). For the preparation of the soluble HIV-1 suppression factor in vitro, the immune human lymphocytes, unseparated, CD4+ T or CD8+ T cells purified by the magnetic beads-positive selection method (Dynal, Oslo, Norway) were cultured in 12-well plates (BD Pharmingen, San Diego, Calif.) in the presence of APC and antigen as stated above.

Assay for Cytokines and Antibodies

Commercial kits for human TNF-alpha, IFN-alpha, IFN-gamma, IL-4, IL-10, IL-12, IL-13, IL-16, MIP-1-alpha, MIP-1-beta, RANTES, MCP-1 and MCP-3 (Biosource, Camarillo, Calif.) TGF-beta (R&D systems, Rockville, Md.) and IFN-beta (Fuji Rebio, Tokyo, Japan) were employed. All assays were performed in accordance with the manufacturer's instructions, and cytokine levels were calculated by comparison to standard curves using recombinant cytokines. For depletion of human beta-chemokines, heparin-Sepharose (Pharmacia, Japan) was used. Bound materials were eluted from the column in 2 M NaCl-containing PBS. For the measurement of OVA specific human antibodies, serial dilutions of serum samples were added to 96-well ELISA microtiter plates (NUNC, Rochester, N.Y.) precoated with 10 μg/ml OVA at 37° C. for 2 h. Bound human antibody was developed with goat anti-human IgG horseradish peroxidase conjugates (American Qualex, San Clemente, Calif.), followed by incubation in a buffer containing tetra-methylbenzidine (Sigma, St. Louis, Mo.) and hydrogen peroxide (Wako pure chemical industries Inc., Osaka, Japan). HIV-1 specific human antibodies were detected by western blot assay using Lav Blot1 (Fiji Rebio co. Ltd., Tokyo, Japan).

Flow Cytometry

For analysis of the CCR5, CXCR4 and CD4, cell samples were incubated in 0.1 mg/ml normal human IgG in FACS buffer (PBS containing 2 % FCS and 0.1% sodium azide) on ice for 15 min, and then stained with FITC- or Cy5-labeled anti-CCR5 (T227) (21), PE-labeled anti-CXCR4 (12G5; BD Pharmingen, San Diego, Calif.), PE-labeled anti-CD4 (Beckman Coulter, Fullerton, Calif.) or Cy5-labeled OKT-4 on ice for 30 min. The cells were washed three times in FACS buffer and fixed with 1% paraformaldehyde in PBS. Cells were analyzed on a flow cytometer, FACS Calibur, using the Cell Quest software (BD Pharmingen, San Diego, Calif.). The area of positivity was determined using an isotype-matched mAbs.

Statistical Analysis

Data were analyzed using Student's t-test by using the Stat View-J 4.02 statistics program (Abacus Concepts, Berkeley, Calif.).

(Results)

Induction of Human Immune Responses in SCID Mice

For the preparation of hu-PBL-SCID mice, SCID mice were generally engrafted with 2×10⁷ fresh human PBMC by ip. injection. In this study, we have attempted an intra-splenic (i.s.) transfer of PBMC, and found that this method was superior to the general one with regard to efficient engraftment with human T cells and reduction of mouse death caused by severe GVHD (data not shown). By using this i.s. transfer method, the number of PBMC required for initial inoculation could be reduced by 1×10⁶ for generation of more than 5×10⁶ human CD3+ T cells within 2 weeks (data not shown). In addition, these mice (hu-PBL-SCID-spl mice) produced higher levels of human Ig than those generated by the ip. transfer. These findings indicate that human T and B lymphocytes inoculated into the mouse spleen are more efficiently activated than those inoculated into the peritoneal cavity.

We tested whether the hu-PBL-SCID-spl mice can generate antigen-specific human immune responses. In the first trials, however, we failed to induce anti-OVA responses by subcutaneous immunization with OVA with Freund's adjuvant (data not shown). In the second trials, we attempted to immunize the mice with antigen-pulsed autologous mature DC generated from peripheral blood monocytes. Fresh PBMC (3×10⁶ cells) from normal donors were transferred into the SCID mouse spleen together with autologous mature DC (5×10⁵ cells) pulsed with OVA (100 μg) or AT-2-inactivated HIV-1_{JR-CSF} (containing 40 ng p24). All the HIV-1 stocks used were prepared in autologous PBMC cultures in order to avoid contamination of allogeneic antigens. On day 5, the mice received an i.s. booster injection with the antigen-pulsed DC (5×10⁵ cells/animal). After 5 days, the mice were examined for antigen-specific human immune responses (FIG. 1). Sera from the DC-OVA-immunized mice showed significant human anti-OVA antibody titer (FIG. 1A), and the lymphocytes from the mice responded to OVA by producing human IFN-gamma upon stimulation with APC in vitro (FIG. 1B). Importantly, the DC-HIV-1-immunized mice showed human anti-HIV-1 cellular immune responses (FIG. 1C). The sera from these mice showed very low or no antibody against HIV-1 as examined by a western blot analysis (data not shown).

Protection Against HIV-1 Challenge in Vivo

In order to examine whether the induced anti-HIV-1 immune responses are protective, these DC-OVA- and DC-HIV-1-immunized hu-PBL-SCID-spl mice were challenged i.p. with infectious R5 HIV-1_{JR-CSF}. After 7 days from the time of infection, mice were examined for HIV-1 infection by assaying for provirus in the lymphocytes and p24 antigen of serum or supernatants of the lymphocyte cultures. Table 1 (see FIG. 1) shows that all the mice immunized with DC-OVA were infected by the HIV-1. Surprisingly, the mice immunized with DC-HIV-1_{JR-CSF} were completely protected against the HIV-1 infection. Furthermore, the mice immunized with inactivated X4 HIV-1_{NL}4-3 were also protected against the R5 HIV-1 infection. Similar results were obtained in the other three experiments using hu-PBL-SCID-spl mice reconstituted with PBMC from two other donors (Table 1 and data not shown). Based on these results, we speculated that the protection of the mice against R5 HIV-1 infection might be mediated by the CCR5 binding human beta-chemokines MIP-1-alpha, MIP-1-beta and/or RANTES. However, the human beta-chemokine levels of the immune sera were lower than those required for suppression of R5 HIV-1 infection in vitro, and the sera from the DC-OVA-immunized mice also contained similar levels of these beta-chemokines (Table 1: see FIG. 1).

Serum Contains a Suppression Factor

The levels of CCR5 and CXCR4 expression on the cell surface of human CD4+ T cells isolated from the DC-HIV-1-immunized mice (protected) were comparable to those from DC-OVA-immunized (unprotected) mice (FIG. 2A). This is in accord with the observation that the CD4+ T cells from protected groups were susceptible to R5 HIV-1 superinfection in vitro (FIG. 2B). These data suggest that the DC-based HIV-1-immunization induces an anti-R5 HIV-1 state in the animals without rendering the human CD4+ T cells intrinsically non-permissive to R5 HIV-1. Thus, we speculated that some soluble HIV-1 suppression factor might be involved.

Pretreatment of R5 HIV-1 virus with the serum samples from the DC-HIV-1-immunized mice did not inhibit the HIV-1 infection (data not shown), suggesting that the factor is not directed against virus itself. Therefore, target PBMC were pretreated with the immune serum samples, and then infected with either R5 or X4 HIV-1, followed by washing and cultivation in IL-2-containing medium. FIG. 3 shows that the sera from either DC-R5 HIV-1 or DC-X4 HIV-1-immunized mice, but not those from DC-OVA-immunized mice, strongly inhibited the productive infection of the PBMC with R5 HIV-1, but not X4 HIV-1, in vitro. As shown in FIG. 4 the DC-HIV-1-immune serum was also suppressive to the other two R5 HIV-1 strains in PBMC, but not to X4 HIV-1 strains. The R5 HIV-1 suppressive activity was not reversed by a mixture of antibodies against the three beta-chemokines (FIG. 4). The factor also suppressed macrophage infection with the three R5 HIV-1 strains, before viral DNA synthesis (FIG. 5). The HIV-1 suppressive activity was neither associated with cell death nor was it MHC-restricted (data not shown).

The R5 suppressive activity of the serum was heat-labile since it was lost by heating at 56° C. for 30 min, suggesting low or no involvement of HIV-1 neutralizing antibody or type-I IFNs. The average levels of human cytokines examined for the sera from the DC-HIV-1-immunized mice were: IFN-alpha, <10 pg/ml; IFN-beta, 20 pg/ml; IL-4, <10 pg/ml; IL-12, <10 pg/ml; IL-13, <10 pg/ml; IL-16, <10 pg/ml; TNF-alpha, <10 pg/ml; and TGF-beta, <10 pg/ml. These data indicate that the serum R5 HIV-1 suppressive activity is mediated by some unknown cytokines of either human or mouse origin.

Human CD4+ T cells Produce the Suppression Factor

In order to define the origin of the suppression factor, human lymphocytes from DC-HIV-1- and DC-OVA-immunized mice were fractionated into human CD4+ T and CD8+ T cell subpopulations by positive selection using antibody-bound magnetic beads, and restimulated in vitro with inactivated HIV-1 and OVA, respectively, in the presence of autologous APC. As shown in FIG. 6A, the human CD4+ T cells from the DC-HIV-1-immunized mice produced the HIV-1 suppression factor, the activity of which was not reversed by the anti-beta-chemokine antibodies. The human CD8+ T cells also produced R5 HIV-1 suppression factor, but the suppressive activity was significantly reversed by the anti-beta-chemokine antibodies. Again, none of these samples were suppressive to X4HIV-1 infection. DC-HIV-1-immune CD4+ T cells obtained from the other hu-PBL-SCID-spl mice transplanted with PBMC from four different donors also produced the HIV-1 suppression factor (FIG. 6B). Since the culture supernatants of OVA-stimulated human CD4+ T cells from the DC-OVA-immunized mice had no or low R5 HIV-1 suppressive activity (data not shown), this suggested that the human CD4+ T cells reactive to HIV-1 antigen are the major producers of the suppression factor.

Partial Characterization of the Suppression Factor

The HIV-1 suppression factor produced in the DC-HIV-1-immune serum and the restimulated CD4+ T cell culture supernatants were further characterized. As shown in FIG. 7A, the suppressive activity was lost by heating at 56° C. for 30 min, was not absorbed to heparin-Sepharose columns, and was not reversed by the anti-beta-chemokine antibodies. Antibody neutralization assay in FIG. 7B shows that the factor was not related to the CCR5-binding beta-chemokines, IL-4, IL-10, IL-12, IL-13, IL-16, MCP-1, MCP-3, TNF-alpha and TNF-beta.

An estimate of the molecular size of the factor was made. The pooled sera from the DC-HIV-1-immunized mice and culture supernatants of the DC-HIV-1 immune CD4+ T cells were depleted of beta-chemokines by the heparin-Sepharose, and fractionated by serial centrifugation over different molecular sheaving filters. FIG. 7C shows that the anti-HIV-1 suppression factor was present in the >100 kDa fraction. Similar results were obtained in the analysis for the CD4+ T cell culture supernatants (data not shown).

Supporting the notion that the present HIV-1 suppression factor does not belong to the CCR5-binding beta-chemokine family, the factor did not down-regulate CCR5 expression on monocytes (FIG. 8A). In addition, as mentioned above, the factor did not affect CD4 expression (FIG. 8B). These observations show that the factor suppresses R5 HIV-1 infection without affecting HIV-1 receptor expression.

(Discussion)

In the present study, we showed for the first time that immunization of the hu-PBL-SCID mice with HIV-1-pulsed mature DC protected the mice against R5 HIV-1 infection, and that the protection was mediated by soluble factor(s) in the serum produced by human CD4+ T cells in response to either R5 or X4 HIV-1 antigen. To elicit the anti-R5 HIV-1 status in the hu-PBL-SCID mice, it was necessary to immunize the mice at least twice with HIV-1-pulsed autologous mature DC by is. injection. The HIV-1 suppression was mediated by a noncytolytic mechanism. It appears that the production of the suppression factor is HIV-1 antigen-dependent, and that close contact between naive CD4+ T cells and HIV-1 antigen presented by DC in the secondary lymphoid organs of mice facilitates the primary human anti-HIV-1 T cell immune responses. The advantage of direct inoculation of SIV antigen into the lymph nodes where mature DCs reside to induce protective immune response has been demonstrated in the simian system (22, 23). Since PBMC specimens from five different healthy individuals produced the HIV-1 suppression factor, it appears that the factor production is not influenced by MHC background. These observations, together with the finding that the suppression factor can be induced in a relatively short period (10 days from the initial immunization) indicate that the present DC-HIV-1 immunization protocol may be useful to induce an immediate protective status in humans.

The induction of primary HIV-1-specific human immune responses in vitro (10, 24) and in the hu-PBL-SCID mice (9, 10) by DC-based immunization has been achieved. In all the cases, the key was the use of matured DC. The type-I IFNs or CD40L have been demonstrated to be DC maturation factors by which the expression of antigen-presenting MHC-class I and II molecules and the costimulatory molecule CD86 are upregulated. Unfortunately, it has not been reported whether the induced anti-HIV-1 T cell immune responses in these studies were protective against HIV-1 infection in vivo. In the present study, we have also confirmed that the use of the HIV-1-pulsed mature DC is essential for the induction of the anti-HIV-1 status in the SCID mice. Because the human CD4+ T cells in the DC-OVA immunized mice did not produce the suppression factor, this suggests that the priming of HIV-1-reactive, naive CD4+ T cells by sufficient numbers of HIV-1-pulsed DC is essential for the factor production in vivo. It remains to be identified which antigen of the HIV-1 particle is responsible for the factor induction. The equal immunogenicity between R5 and X4 HIV-1 virions in the induction of the HIV-1 immunity suggests that the V3 region of the env gp120 and viral nonstructural proteins are not involved. Further studies on the reactivity of the HIV-1-immune CD4+ T cells against a panel of synthetic peptides of various HIV-1 proteins will help determine the identity of the immunogenic antigen. From a therapeutic aspect, it will be of interest to determine whether the factor can be produced from CD4+ T cells from HIV-1-infected individuals upon HIV-1 antigen stimulation in vivo and in vitro.

The mechanism for HIV-1 suppression by the present factor appears to be the inhibition of an early stage of virus infection. The preferential suppression of R5 HIV-1 in activated primary PBMC cultures and macrophages in vitro suggests that the factor may belong to CCR5-binding beta-chemokines. However, the facts that the concentrations of MIP-1-alpha, MIP-1-beta and RANTES of the DC-HIV-1-immune serum samples or the in vitro restimulated cell culture supernatants were too low to suppress HIV-1 in vitro, that the neutralizing antibodies against the three human beta-chemokines did not reverse the suppressive activity, and that the suppression factor was not absorbed by the heparin-Sepharose column, strongly suggest that the factor is not related to the beta-chemokines. Furthermore, the unchanged expression of CCR5 on macrophages or CD4+ T cells after treatment with the factor, diminishes the possible relationship between the factor and the CCR5-binding beta-chemokines. The other cytokines known to suppress R5 HIV-1 proliferation in primary macrophages are the Th2 cytokines, IL-4 and IL-10 (25), and the proinflammatory cytokines TNF-alpha (26) and IFN-gamma (27, 28). However, the involvement of these cytokines for the present R5 HIV-1 suppression is less likely since the blocking antibodies against these cytokines did not interfere with the suppressive activity in the present assay conditions. The involvement of human type-I IFNs, of which anti-HIV-1 protective activity in the SCID mice has been demonstrated (29), is unlikely either, since the X4 HIV-1 infection of PBMC was not suppressed by the factor under the same experimental conditions. Another possibility is that anti-HIV-1 neutralizing antibodies and/or murine complements are involved. However, their contribution in vivo will be minimal, since of the suppressive activity of the HIV-1-immune serum was heat labile and fresh sera from the DC-OVA immune mice had no suppressive activity.

The characteristics of the present HIV-1 suppression factor are also different from other, as yet undefined, HIV-1 suppression factors. First, the present factor is mainly, but not exclusively, produced by DC-HIV-1 immune CD4+ T cells, while the so-called CD8 antiviral factor (CAF) is produced by CD8+ T cells of HIV-1-infected individuals, and CAF inhibits both R5 and X4 HIV-1 production at the level of viral transcription (30). It is important to note that the hu-PBL-SCID mice reconstituted with human PBMC of HIV-1-exposed but uninfected individuals were resistant to both R5 and X4 HIV-1 infection by a CD8+ T cell-dependent mechanism. However, it has not been reported whether the anti-HIV-1 effect was mediated by the CCR5-binding chemokines or CAF. The high molecular size of the present factor argues against a relationship to the human defensin-a1, -2 and - 3, which have recently been demonstrated to be produced by human CD8+ T cells and block both R5 and X4 HIV-1 infection of activated PBMC (31). Another CAF candidate is the modified form of bovine antithrombin III that is produced by CD8+ T cells of HIV-1 infected individuals (32). This molecule is heat-stable, 40 kDa in gel filtration, suppressive to both X4 and R5 HIV-1 infection of cell lines, and thus clearly different from the present factor. Similarly, differences in molecular size and HIV-1 selectivity in suppression suggests that the present factor is distinct from the secretory leukocyte protease inhibitor that is a potent anti-HIV-1 factor in saliva (33), and soluble polyanions such as dextran sulfate, heparin, or heparan sulfate, which interfere with CD4− and coreceptor-independent HIV-1 attachment to the cell surface heparan sulfate proteoglycans (34). It has been shown that the induction of beta-chemokine-independent intrinsic resistance of CD4+ T cells to R5 HIV-1 infection can be achieved in vitro by strong stimulation of CD4+ T cells with a combination of anti-CD3 and anti-CD28 (35). Furthermore, it has been shown that naive, but not memory, CD4+ T cells from HIV-1-negative donors become resistant to R5 HIV-1 upon dual stimulation with anti-CD3 mAb and either anti-CD28 mAb or CD80 independently of CCR5-binding chemokines (36). Although these CD4+ T cells could suppress R5 HIV-1 replication in activated memory CD4+ T cells, it remains unclear whether these stimulated naive CD4+ T cells secrete HIV-1 suppression factors identical to the present factor.

In conclusion, the present study has demonstrated for the first time that a DC-based HIV-1 vaccination can induce HIV-1-reactive human CD4+T cells which produce a yet-undefined R5 HIV-1 suppression factor in the hu-PBL-SCID mice, and thus suggests a rational basis for the DC-based immunization against HIV-1 infection in humans.

### REFERENCES

1. McCune, J., H. Kaneshima, J. Krowka, R. Namikawa, H. Outzen, B. Peault, L. Rabin, C. C. Shih, E. Yee, M. Lieberman, and et al. 1991. The SCID-hu mouse: a small animal model for HIV infection and pathogenesis. Annu. Rev Immunol. 9:399.

2. Mosier, D. E. 1996. Human immunodeficiency virus infection of human cells transplanted to severe combined immunodeficient mice. Adv Immunol. 63:79.

3. Rizza, P., S. M. Santini, M. A. Logozzi, C. Lapenta, P. Sestilh, G. Gherardi, R. Lande, M. Spada, S. Parlato, F. Belardelli, and S. Fais. 1996. T-cell dysfunctions in hu-PBL-SCID mice infected with human immunodeficiency virus (HIV) shortly after reconstitution: in vivo effects of HIV on highly activated human immune cells. J. Virol. 70:7958.

4. Tary-Lehmann, M., A. Saxon, and P. V. Lehmann. 1995. The human immune system in hu-PBL-SCID mice. Immunol. Today 16:529.

5. Mazingue, C., F. Cottrez, C. Auriault, J. Y. Cesbron, and A. Capron. 1991. Obtention of a human primary humoral response against schistosome protective antigens in severe combined immunodeficiency mice after the transfer of human peripheral blood mononuclear cells. Eur. J. Immunol. 21:1763.

6. Aaberge, I. S., T. E. Steinsvik, E. C. Groeng, R. B. Leikvold, and M. Lovik. 1996. Human antibody response to a pneumococcal vaccine in SCID-PBL-hu mice and simultaneously vaccinated human cell donors. Clin. Exp. Immunol. 105:12.

7. Ifversen, P., C. Martensson, L. Danielsson, C. Ingvar, R. Carlsson, and C. A. Borrebaeck. 1995. Induction of primary antigen-specific immune reponses in SCID-hu-PBL by coupled PB epitopes. Immunology 84:111.

8. Bombil, F., J. P. Kints, J. M. Scheiff, H. Bazin, and D. Latinne. 1996. A promising model of primary human immunization in human-scid mouse. Immunobiology 195:360.

9. Delhem, N., F. Hadida, G. Gorochov, F. Carpentier, J. P. de Cavel, J. F. Andreani, B. Autran, and J. Y. Cesbron. 1998. Primary Th1 cell immunization against HIVgp160 in SCID-hu mice coengrafted with peripheral blood lymphocytes and skin. J. Immunol. 161.2060.

10. Santini, S. M., C. Lapenta, M. Logozzi, S. Parlato, M. Spada, T. Di Pucchio, and F. Belardelli. 2000. Type I interferon as a powerful adjuvant for monocyte-derived dendritic cell development and activity in vitro and in Hu-PBL-SCID mice. J. Exp. Med. 191:1777

11. Mosier, D. E., R. J. Gulizia, S. M. Baird, D. B. Wilson, D. H. Spector, and S. A. Spector. 1991. Human immunodeficiency virus infection of human-PBL-SCID mice. Science 251:791.

12. Ito, M., H. Hiramatsu, K. Kobayashi, K. Suzue, M. Kawahata, K. Hioki, Y. Ueyama, Y. Koyanagi, K. Sugamura, K. Tsuji, T. Heike, and T. Nakahata. 2002. NOD/SCID/gamma mouse: an excellent recipient mouse model for engraftment of human cells. Blood 100:3175.

13. Ohteki, T., T. Fukao, K. Suzue, C. Maki, M. Ito, M. Nakamura, and S. Koyasu. 1999. Interleukin 12-dependent interferon gamma production by CD8a+ lymphoid dendritic cells. J. Exp. Med. 189:1981.

14. Tanaka, T., F. Kitamura, Y. Nagasaka, K. Kuida, H. Suwa, and M. Miyasaka. 1993. Selective long-term elimination of natural killer cells in vivo by an anti-interleukin 2 receptor beta chain monoclonal antibody in mice. J. Exp. Med. 178:1103.

15. Koyanagi, Y., S. Miles, R. T. Mitsuyasu, J. E. Merrill, H. V. Vinters, and I. S. Chen. 1987. Dual infection of the central nervous system by AIDS viruses with distinct cellular tropisms. Science 236:819.

16. Adachi, A., H. E. Gendelman, S. Koenig, T. Folks, R. Willey, A. Rabson, and M. A. Martin. 1986. Production of acquired immunodeficiency syndrome-associated retrovirus in human and nonhuman cells transfected with an infectious molecular clone. J. Virol. 59:284.

17. Subramani, S., R. Mulligan, and P. Berg. 1981. Expression of the mouse dihydrofolate reductase complementary deoxyribonucleic acid in simian virus 40 vectors. Mol Cell Biol 1:854

18. Shioda, T., J. A. Levy, and C. Cheng-Mayer. 1991. Macrophage and T cell-line tropisms of HIV-1 are determined by specific regions of the envelope gp120 gene. Nature 349:167.

19. Rossio, J. L., M. T. Esser, K. Suryanarayana, D. K. Schneider, J. W. Bess, Jr., G. M. Vasquez, T. A. Wiltrout, E. Chertova, M. K. Grimes, Q. Sattentau, L. O. Arthur, L. E. Henderson, and J. D. Lifson. 1998. Inactivation of human immunodeficiency virus type 1 infectivity with preservation of conformational and functional integrity of virion surface proteins. J. Virol. 72:7992.

20. Koyanagi, Y., Y. Tanaka, J. Kira, M. Ito, K. Hioki, N. Misawa, Y. Kawano, K. Yamasaki, R. Tanaka, Y. Suzuki, Y Ueyama, E. Terada, T. Tanaka, M. Miyasaka, T. Kobayashi, Y. Kumazawa, and N. Yamamoto. 1997. Primary human immunodeficiency virus type 1 viremia and central nervous system invasion in a novel hu-PBL-immunodeficient mouse strain. J. Virol. 71:2417

21. Suzuki, Y., Y. Koyanagi, Y. Tanaka, T. Murakami, N. Misawa, N. Maeda, T. Kimura, H. Shida, J. A. Hoxie, W. A. O'Brien, and N. Yamamoto. 1999. Determinant in human immunodeficiency virus type 1 for efficient replication under cytokine-induced CD4+ T-helper 1 (Th1)- and Th2-type conditions. J. Virol. 73:316.

22. Lehner, T., Y. Wang, M. Cranage, L. A. Bergmeier, E. Mitchell, L. Tao, G. Hall, M. Dennis, N. Cook, R. Brookes, L. Klavinskis, I. Jones, C. Doyle, and R. Ward. 1996. Protective mucosal immunity elicited by targeted iliac lymph node immunization with a subunit SIV envelope and core vaccine in macaques. Nat. Med. 2:767.

23. Lehner, T., L. A. Bergmeier, L. Tao, C. Panagiotidi, L. S. Klavinskis, L. Hussain, R. G. Ward, N. Meyers, S. E. Adams, A. J. Gearing, and et al. 1994. Targeted lymph node immunization with simian immunodeficiency virus p27 antigen to elicit genital, rectal, and urinary immune responses in nonhuman primates. J. Immunol. 153:1858.

24. Zhao, X. Q., X. L. Huang, P. Gupta, L. Borowski, Z. Fan, S. C. Watkins, E. K. Thomas, and C. R. Rinaldo, Jr. 2002. Induction of anti-human immunodeficiency virus type 1 (HIV-1) CD8+ and CD4+ T-cell reactivity by dendritic cells loaded with HIV-1 X4-infected apoptotic cells. J. Virol. 76:3007

25. Montaner, L. J., and S. Gordon. 1995. TH2 downregulation of macrophage HIV-1 replication. Science 267:538.

26. Lane, B. R., D. M. Markovitz, N. L. Woodford, R. Rochford, R. M. Strieter, and M. J. Coffey. 1999. TNF-alpha inhibits HIV-1 replication in peripheral blood monocytes and alveolar macrophages by inducing the production of RANTES and decreasing C—C chemokine receptor 5 (CCR5) expression. J. Immunol. 163:3653.

27. Dhawan, S., L. M. Wahl, A. Heredia, Y. Zhang, J. S. Epstein, M. S. Meltzer, and I. K. Hewlett. 1995. Interferon-gamma inhibits HIV-induced invasiveness of monocytes. J Leukoc. Biol. 58:713.

28. Zaitseva, M., S. Lee, C. Lapham, R. Taffs, L. King, T. Romantseva, J. Manischewitz, and H. Golding. 2000. Interferon gamma and interleukin 6 modulate the susceptibility of macrophages to human immunodeficiency virus type 1 infection. Blood 96:3109.

29. Lapenta, C., S. M. Santini, E. Proietti, P. Rizza, M. Logozzi, M. Spada, S. Parlato, S. Fais, P. M. Pitha, and F. Belardelli. 1999. Type I interferon is a powerful inhibitor of in vivo HIV-1 infection and preserves human CD4+ T cells from virus-induced depletion in SCID mice transplanted with human cells. Virology 263:78.

30. Levy, J. A., C. E. Mackewicz, and E. Barker. 1996. Controlling HIV pathogenesis: the role of the noncytotoxic anti-HIV response of CD8+ T cells. Immunol. Ybday 17:217

31. Zhang, L., W. Yu, T. He, J. Yu, R. E. Caffrey, E. A. Dalmasso, S. Fu, T. Pham, J. Mei, J. J. Ho, W. Zhang, P. Lopez, and D. D. Ho. 2002. Contribution of human alpha-defensin-1, -2 and -3 to the Anti-HIV-1 Activity of CD8 Antiviral Factor. Science 26.26.

32. Geiben-Lynn, R., M. Kursar, N. V. Brown, E. L. Kerr, A. D. Luster, and B. D. Walker. 2001. Noncytolytic inhibition of X4 virus by bulk CD8+ cells from human immunodeficiency virus type 1 (HIV-1)-infected persons and HIV-1-specific cytotoxic T lymphocytes is not mediated by beta-chemokines. J. Virol. 75:8306.

33. McNeely, T. B., D. C. Shugars, M. Rosendahl, C. Tucker, S. P. Eisenberg, and S. M. Wahl. 1997. Inhibition of human immunodeficiency virus type 1 infectivity by secretory leukocyte protease inhibitor occurs prior to viral reverse transcription. Blood 90:1141.

34. Saphire, A. C., M. D. Bobardt, Z. Zhang, G. David, and P. A. Gallay. 2001. Syndecans serve as attachment receptors for human immunodeficiency virus type 1 on macrophages. J. Virol. 75:9187.

35. Riley, J. L., R. G. Carroll, B. L. Levine, W. Bernstein, D. C. St Louis, 0. S. Weislow, and C. H. June. 1997. Intrinsic resistance to T cell infection with HIV type 1 induced by CD28 costimulation. J. Immunol. 158:5545.

36. Mengozzi, M., M. Malipatlolla, S. C. De Rosa, L. A. Herzenberg, and M. Roederer. 2001. Naive CD4 T cells inhibit CD28-costimulated R5 HIV replication in memory CD4 T cells. Proc. Natl. Acad. Sci. USA 98:11644.

Abbreviations used herein: DC, dendritic cells; human PBMC transplanted SCID mouce; AT2, aldorichol-2; TCID₅₀, tissue culture infectious dose; hu-PBL-SCID, human peripheral blood lymphocytes transferred severe combined immunodeficiency mouse; i. s., intra-splenic; CAF, CD8 antiviral factor.

What is claimed:

1. A vaccine composition comprising autologous mature dendritic cells (DC) pulsed with inactivated human immunodeficiency virus (HIV).

2. The vaccine composition of claim 1, wherein said HIV is HIV-1.

3. The vaccine composition of claim 2, wherein said HIV-1 is R5 HIV-1_{JR-CSF}.

4. The vaccine composition of claim 1, wherein said DC is derived from peripheral blood mononuclear cells (PBMC).

5. The vaccine composition of claim 1, wherein said DC is prepared by culturing the PBMC in medium containing granulocyte-macrophage colony stimulating factor (GM-CSF) and human interleukin-4 (IL-4).

6. The vaccine composition of claim 5, wherein the PBMC is further cultured in medium containing human interferon-beta (IFN-beta).

7. An HIV-infection suppression factor which is produced by human CD4+ pulsed with inactivated HIV, has a molecule weight of more than 100 kDa, is not absorbed to heparin-Sepharose columns, and is inactivated by heating at 56 degree Celsius for 30 min.

8. The HIV-infection suppression factor of claim 7, wherein the HIV is HIV-1.

9. The HIV-infection suppression factor of claim 7, wherein the HIV is R5 HIV1_{JR-CSF}.

10. The HIV-infection suppression factor of claim 7, wherein the factor is not lost its suppression activity by subjecting to neutralizing antibodies against human RANTES, MIP-1-alpha, MIP-1-beta, IFN-alpha, IFN-beta, IFN-gamma, IL-4, IL-10, IL-13, IL-16, MCP-1, MCP-3, TNF-alpha or TNF-beta.

11. The HIV-infection suppression factor of claim 7, wherein the factor is not lost its suppression activity by subjecting to anti-beta-chemokine antibodies.

12. A method for the vaccination against HIV comprising administering autologous mature DC pulsed with inactivated HIV to a subject to be vaccinated.

13. The method of claim 12, wherein said HIV is HIV-1.

14. The method of claim 13, wherein said HIV-1 is R5 HIV-1_{JR-CSF}.

15. A method for the vaccination against HIV comprising: collecting PBMC from a subject to be vaccinated, culturing the PBMC in medium containing GM-CSF and human IL-4 to prepare autologous mature DC, pulsing the DC with inactivated HIV, and administering the DC to the subject.

16. The method of claim 15, which further includes culturing the PBMC in medium containing IFN-beta.

17. The method of claims 15 or 16, wherein said HIV is HIV-1.

18. The method of claims 17, wherein said HIV-1 is R5 HIV-1_{JR-CSF}.
